(19) United States
(12) Patent Application Publication
(10) Pub. No.: US 2005/0215888 A1
(43) Pub. Date: Sep. 29, 2005

(54) Universal support arm and tracking array

(76) Inventors: James E. Grimm, Winona Lake, IN (US); Shawn E. McGinley, Fort Wayne, IN (US); Jacque R. Wilson, Fort Wayne, IN (US); Maleata Y. Hall, Warsaw, IN (US); Dale E. Walriven, Warsaw, IN (US); Sudip Hui, Warsaw, IN (US); Chetan Rangaiah, Warsaw, IN (US)

Correspondence Address: BAKER & DANIELS LLP;111 E. WAYNE STREET, SUITE 800, FORT WAYNE, IN, 46802, US

(21) Appl. No.: 11/057,593

(22) Filed: Feb. 14, 2005

Related U.S. Application Data
(60) Provisional application No. 60/550,460, filed on Mar. 5, 2004.

(51) Int. Cl. A61B 5/05

(52) U.S. Cl. 600/426; 606/130

(57) ABSTRACT

A universal tracking apparatus for a surgical instrument, the tracking apparatus including a support arm and tracking array. The support arm allows position adjustment of the tracking array between a number of predefined orientations relative to the instrument which are recognizeable and registerable in the navigation system. The support arm may be coupled with multiple types of surgical instruments and used to accurately define the position of the instrument in an anatomical model generated by the navigation system. The adjustable coupling between the tracking array and the support arm allows the surgeon to determine which orientation is best suited for the surgical application and also allows the surgeon to adjust the position of the tracking array during a surgical procedure without the need to re-register the tracking apparatus. The tracking array may also include at least one repositionable reference element to allow a single tracking array to be configured for use with a plurality of different instruments, wherein differing geometries defined by the tracking array and corresponding to particular instruments are recognizable and registerable by the navigation system.

### CROSS REFERENCE TO RELATED APPLICATIONS

This application is related to and claims the benefit under Title 35, U.S.C. §119(e) of U.S. Provisional Patent Application Ser. No. 60/550,460, entitled UNIVERSAL SUPPORT ARM AND TRACKING ARRAY, filed on Mar. 5, 2004.

### BACKGROUND OF THE INVENTION

1. Field of the Invention

The present invention relates to computer-assisted navigation for use in surgical procedures and, more specifically, to a tracking apparatus including a support arm which may be detachably coupled to a surgical instrument, the support arm including a tracking array for use in accurately locating the position of the surgical instrument during a surgical procedure.

2. Description of the Related Art

Various methods and guide instruments have been developed to facilitate the proper positioning of a surgical instrument during a surgical procedure. Such instruments and methods include the use of mechanical surgical guides which can be positioned in relation to one or more anatomical structures to function as mechanical guides for cutting, reaming, and drilling instruments, for example. For example, these types of mechanical guides may be used to locate and align a surgical instrument with respect to a bone when preparing the bone for receiving an implant, such as a component of an artificial joint.

Computer-assisted navigation systems are well known, and can also be used to facilitate proper instrument placement during a surgical procedure. Computer-assisted navigation techniques often involve acquiring preoperative images of the relevant anatomical structures of a patient, and generating an anatomical coordinate system database that represents a three-dimensional model of the anatomical structures. The relevant surgical instruments typically have a known and fixed geometry that is also defined in the database preoperatively.

During the surgical procedure, the position of the instrument being used, as well as the location of one or more relevant anatomical landmarks, are registered with the anatomical coordinate system. Generally, registration is the process of relating pre-procedural or intra-procedural scans of the relevant anatomy and/or data corresponding to an instrument, to the surgical or medical position of the corresponding anatomy and/or instrument. A graphical display showing the relative position of the instrument with respect to the relevant anatomical structures may then be computed in real time and displayed for the surgeon to assist in properly positioning and manipulating the surgical instrument with respect to the relevant anatomical structures. In such image-guided procedures, a robotic arm may be used to position and control the instrument, or alternatively, the surgeon may manually position the instrument using the display of the relative position of the instrument with respect to the anatomical structures to facilitate proper positioning of the instrument.

Surgical instruments have typically been adapted for use with computer-assisted navigation systems by clamping a fixed tracking array that is registrable in the navigation system onto the surgical instrument at an arbitrary location and orientation relative to the instrument. Because the location and orientation is arbitrary, the geometric relationship between the tracking array and the surgical instrument must then be calibrated in order to register the combination of the tracking array and the surgical instrument in the navigation system and to properly graphically display the relative position and orientation of the instrument with respect to the anatomical structures. Thus, each time the tracking array is coupled to a surgical instrument, the combination must again be carefully calibrated and registered to ensure that the graphical display viewed by the surgeon on the computer-assisted navigation system accurately reflects the true position of the instrument relative to the anatomical structures.

The variability associated with arbitrarily clamping a tracking array to a surgical instrument, as well as the possibility of a clamped tracking array slipping relative to the surgical instrument, causes uncertainty in the geometry of the combination, thus requiring careful and possibly repeated instrument calibrations. Surgical instruments including a tracking array permanently fixed to the instrument eliminate this uncertainty. However, a surgical instrument so equipped may not be suitable for procedures which require the array to be in a different position relative to the instrument, or may not be suitable for use without the computer-assisted navigation system because of the permanently-affixed tracking array.

After use of a surgical instrument having a tracking array clamped thereto has begun, it may become necessary to reposition the tracking array relative to the surgical instrument. For example, the position of the tracking array may present a barrier to accessing the anatomical structures visually or with other instruments, or the position of the tracking array may need to be adjusted to place the tracking array properly in the “line of sight” of, or otherwise within the applicable field of detection of, the computer-assisted navigation system. Repositioning the tracking array may require re-clamping the tracking array to the instrument, followed by recalibration and re-registration of the assembly with the computer-assisted navigation system.

Also, when multiple surgical instruments are independently positioned relative to the anatomical structures, different tracking arrays each having a different pattern of reference elements may be clamped to the instruments, thus enabling the computer-assisted navigation system to distinguish between the different tracking arrays and in turn the different instruments. Disadvantageously, such a system requires a supply of many different tracking arrays for each surgical procedure.

What is needed is a tracking apparatus for use in a computer-assisted navigation system which is an improvement over the foregoing.

### SUMMARY OF THE INVENTION

The present invention provides a universal tracking apparatus for a surgical instrument, the tracking apparatus including a support arm and tracking array. The support arm allows position adjustment of the tracking array between a number of predefined orientations relative to the instrument which are recognizeable and registerable in the navigation system. The support arm may be coupled with multiple types of surgical instruments and used to accurately define the position of the instrument in an anatomical model generated by the navigation system. The adjustable coupling between the tracking array and the support arm allows the surgeon to determine which orientation is best suited for the surgical application and also allows the surgeon to adjust the position of the tracking array during a surgical procedure without the need to re-register the tracking apparatus. The tracking array may also include at least one repositionable reference element to allow a single tracking array to be configured for use with a plurality of different instruments, wherein differing geometries defined by the tracking array and corresponding to particular instruments are recognizable and registerable by the navigation system.

In one exemplary embodiment, the tracking apparatus of the present invention includes a support arm having first, second, and third members. The first member includes a mounting interface for releasably coupling the first member to a surgical instrument in a predefined geometric relationship with respect to the support arm. The second member is moveable between a plurality of predefined positions relative to the first member to permit adjustment of the support arm. For example, the second member may be rotationally coupled with the first member about a first axis, wherein the second member is rotatable relative to the first member about the first axis between a plurality of predefined positions. Also, the third member is moveable between a plurality of predefined positions relative to the second member to permit further adjustment of the support arm. For example, the third member may be rotationally coupled with the second member about a second axis which is substantially perpendicular to the first axis for rotational adjustment about the second axis between a plurality of predefined positions.

The third member includes a mounting interface for coupling a tracking array which may be registered in the computer-assisted navigation system. By repositioning the second and third members to selected predefined positions, the position of the tracking array relative to the instrument may be adjusted. Also, at least one of the first, second, and third members may include a reference indicator registerable in the navigation system, such that after the tracking apparatus is adjusted, the orientation of the tracking apparatus may be automatically recognized by the navigation system, eliminating the need for the surgeon to manually re-register the tracking apparatus in the navigation system.

In one exemplary embodiment, the first and/or third members may be coupled to the second member so that they may be rotated to a limited number of predefined positions relative to the second member, thereby simplifying the task of indicating to the computer-assisted navigation system the relative geometry between the instrument and the tracking array. For example, a set of matching protuberances and recesses may be located on adjacent members and the members biased by a spring to engage the protuberances in the recesses. To reposition the first or third member relative to the second member, the members may be pulled slightly apart against the force of a spring to disengage the protuberances from the recesses and then rotated to another predefined position in which the protuberances may again engage matching recesses. The selected predefined position may then be input manually to the computer-assisted navigation system, or a reference element may be coupled to the first member in order for the computer-assisted navigation system to determine the geometry between the tracking array and first member, thereby defining the geometry between the tracking array and instrument.

In another exemplary embodiment, the tracking apparatus includes a tracking array usable for tracking an instrument in a computer-assisted navigation system. The tracking array includes a body member, at least three reference elements coupled with the body member in a nonlinear pattern, and a mounting interface coupled with the body member for coupling the body member with an instrument. At least one of the reference elements is adjustable in its position relative to the body member. For example, the reference elements may be coplanar, with one reference element movable within the plane relative to the remaining reference elements, thus forming a different reference element pattern. The pattern may be distinct from other patterns and/or other tracking arrays used with the computer-assisted navigation system in order to uniquely identify the tracking array, and the instrument to which it is coupled, from the other tracking arrays and instruments.

The reference elements of the exemplary tracking array may be passive elements such as reflective spheres, for example, the positions of which are detectable by a position sensor of the navigation system. Alternatively, the reference elements may be active elements which emit a signal detectable by a position sensor of the navigation system.

The adjustable or repositionable reference element may be repositioned by providing, for example, multiple mounting interfaces to which the reference element may be coupled, or by providing a translating, pivoting, or otherwise movable mounting interface which is coupled to the body of the tracking array and upon which the reference element is mounted. The mechanism for moving the mounting interface relative to the body may include detents or other mechanical devices to provide predetermined positions. Optionally, a template may be used for accurately repositioning the movable reference element relative to the other reference elements. The template may include recesses corresponding to the fixed reference elements and the multiple positions of the movable reference element.

Advantageously, the support arm or tracking array may be repositioned to provide an optimal view of the tracking array by the position sensor, or to provide unobstructed access for the surgeon to the anatomical structures of the patient without having to uncouple the support arm from the instrument or having to re-register the tracking apparatus.

Further, once an instrument is positioned and secured relative to the anatomical structures of the patient, the support arm and tracking array may be detached from a surgical instrument, providing increased access to the instrument and anatomical structure. Additionally, the flexibility of adjusting the pivotable couplings of the support arm and the movable reference element of the tracking array reduces the number of different components that need be available to perform a surgical procedure.

In one form thereof, the present invention provides a tracking apparatus for use with a surgical instrument in a computer-assisted surgical navigation system, including a support arm, including a first member having a surgical instrument mounting interface, and a second member adjustably coupled to the first member; and a tracking array adjustably coupled to the second member, the tracking array including at least one reference element which is registerable in the navigation system.

In another form thereof, the present invention provides a tracking apparatus for use with a surgical instrument in a computer-assisted surgical navigation system, including a support arm including a surgical instrument mounting interface; and a tracking array adjustably coupled to the support arm, the tracking array including a plurality of reference elements which are registerable in the navigation system, at least one of the reference elements adjustably coupled to the tracking array whereby the relative position of the at least one reference element with respect to others of the reference elements may be varied.

In a further form thereof, the present invention provides a tracking array for use with a surgical instrument in a computer-assisted surgical navigation system, including a body member; and a plurality of reference elements coupled to the body member, the reference elements registerable in the navigation system, at least one reference element adjustably coupled to the body member, whereby the relative position of the at least one reference element with respect to others of the reference elements may be varied.

In a further form thereof, the present invention provides a method of using a tracking apparatus in a computer-assisted surgical navigation system, including the steps of providing a surgical instrument; providing a tracking apparatus including a support arm having first and second members adjustably coupled to one another, and a tracking array adjustably coupled to the second member, the tracking array registerable in the navigation system; coupling a surgical instrument to the first member of the support arm; registering the tracking apparatus in the navigation system; adjusting at least one of the position of the second member with respect to the first member, and the position of the tracking array with respect to the second member; and re-registering the tracking apparatus with the navigation system.

In the foregoing method, the re-registering step may occur automatically without manual intervention by an operator. Also, the adjusting step may include at least one of rotationally adjusting the position of the second member with respect to the first member about a first axis; and rotationally adjusting the position of the tracking array with respect to the second member about a second axis different from the first axis. Further, the tracking array may include a plurality of reference elements registerable in the navigation system, the method further including the additional step, prior to the registering step, of adjusting the position of at least one reference element of the tracking array with respect to at least one other reference element of the tracking array. Still further, the coupling step may include threading a threaded member associated with one of the support arm and the instrument into a threaded bore of the other of the support arm and the instrument; and inserting a locating member associated with one of the support arm and the instrument into a locating recess in the other of the support arm and the instrument.

In a still further form thereof, the present invention provides a method of using a tracking apparatus in a computer-assisted surgical navigation system, including the steps of providing a surgical instrument; providing a tracking apparatus including a plurality of reference elements registerable in the navigation system; coupling the surgical instrument to the tracking apparatus; adjusting the position of at least one of the reference elements with respect to others of the reference elements; and registering the tracking apparatus with the navigation system.

In the foregoing method, the adjusting step may further include adjusting the position of at least one of the reference elements with respect to others of the reference elements to define a predetermined reference element geometry corresponding to the surgical instrument. Also, the adjusting step may further include positioning the tracking apparatus with respect to a template; and securing the at least one reference element in a position defined by the template. Further, the coupling step may include threading a threaded member associated with one of the support arm and the instrument into a threaded bore of the other of the support arm and the instrument; and inserting a locating member associated with one of the support arm and the instrument into a locating recess in the other of the support arm and the instrument. Still further, the method may include the additional steps of decoupling the surgical instrument from the tracking apparatus; coupling a different surgical instrument to the tracking apparatus; adjusting the position of at least one of the reference elements with respect to others of the reference elements; and re-registering the tracking apparatus with the navigation system.

In a still further form thereof, the present invention provides a tracking apparatus for use with a surgical instrument in a computer-assisted surgical navigation system, including a support arm including a surgical instrument mounting interface; a tracking array adjustably coupled to the support arm, the tracking array including at least one reference element which is registerable in the navigation system; and the support arm and the tracking array further including cooperating reference indicators registerable in the navigation system for determining a relative orientation between the support arm and the tracking array.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a perspective assembly view of a tracking apparatus, including a support arm coupling a surgical instrument with a tracking array in accordance with the present invention;

FIG. 2 is an exploded perspective view of the components of the tracking apparatus of FIG. 1;

FIG. 3 is a perspective view of an operating room arrangement having a computer-assisted navigation system utilizing the tracking apparatus of FIG. 1;

FIG. 4 is a side view of a registration probe for use with the support arm of the tracking apparatus of FIG. 1;

FIG. 5A is a top perspective view of a second exemplary tracking array in accordance with the present invention;

FIG. 5B is an end view of the tracking array of FIG. 5A;

FIG. 6 is a top perspective view of a third exemplary tracking array in accordance with the present invention;

FIG. 7A is a plan view of a first exemplary template for use with a tracking array of the present invention;

FIG. 7B is a top view of a tracking array of the present invention coupled with the first exemplary template of FIG. 7A;

FIG. 8 is a plan view of second exemplary template which may be used with a tracking array of the present invention;

FIG. 9 is a schematic representation of exemplary method steps for using the exemplary tracking apparatus of FIGS. 1 and 2; and

FIG. 10 is a schematic representation of exemplary method steps for using the second and third exemplary tracking assemblies of FIGS. 5 and 6.

Corresponding reference characters indicate corresponding parts throughout the several views. Although the drawings represent embodiments of the present invention, the drawings are not necessarily to scale and certain features may be exaggerated to better illustrate and explain the present invention. The exemplifications set out herein illustrate embodiments of the invention, in several forms, and such exemplifications are not to be construed as limiting the scope of the invention in any manner.

### DETAILED DESCRIPTION

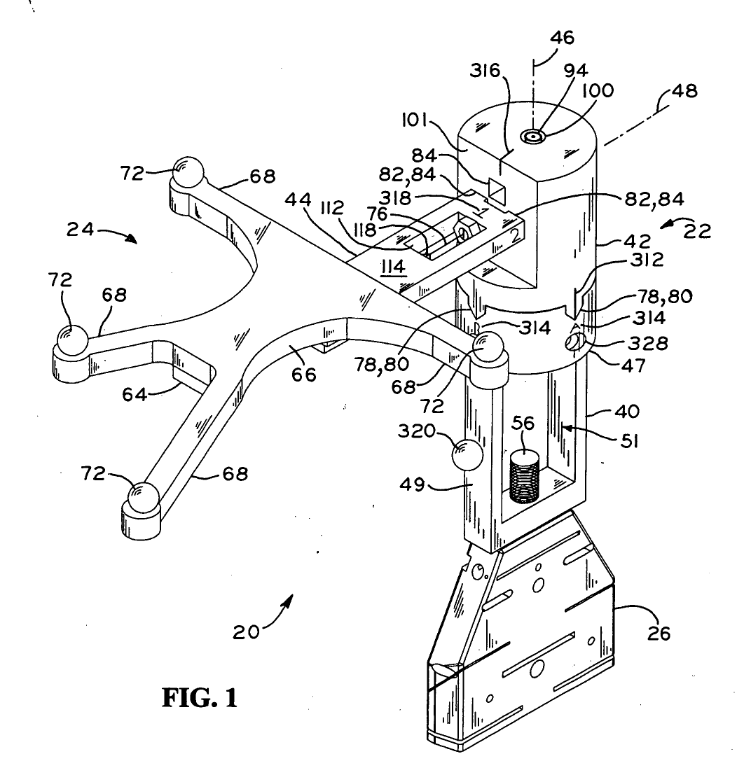

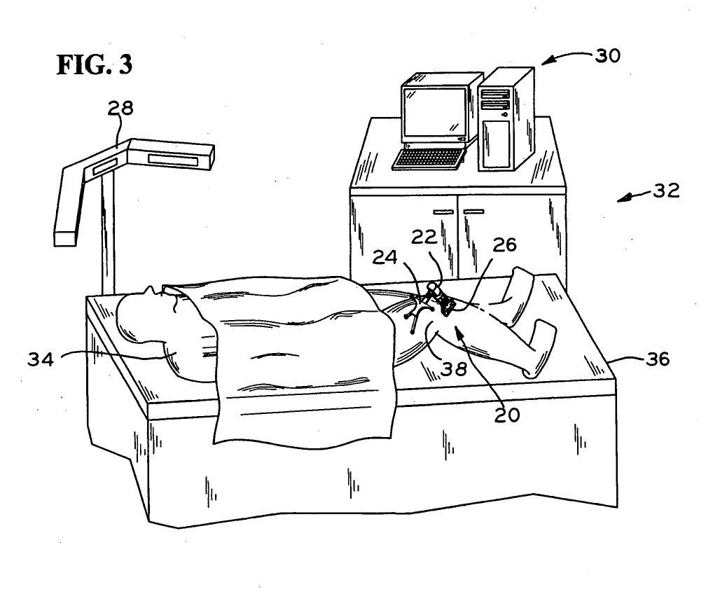

Referring initially to FIG. 1, an exemplary tracking apparatus 20 according to the present invention includes support arm 22 for coupling tracking array 24 to surgical instrument 26. Support arm 22 displaces tracking array 24 from instrument 26, for example for placing array 24 within the field of view or detection of position sensing unit 28 of computer-assisted navigation system 30, shown in FIG. 3. Additionally, support arm 22 allows positional adjustment of tracking array 24 relative to instrument 26, thereby providing adjustment of the geometry between instrument 26 and array 24. Instrument 26 may be any instrument used with navigation system 30, for example a cut guide for orthopedic implant surgery, or a saw, reamer, drill, or other surgical instrument.

Referring to FIG. 3, operating room arrangement 32 includes computer-assisted navigation system 30, position sensing unit 28 for detecting the position of tracking array 24 of tracking apparatus 20, and patient 34, who is positioned on operating table 36. Navigation system 30 assists a surgeon in positioning instrument 26 relative to anatomical structures of patient 34, for example anatomical structures of knee 38 when performing knee arthroplasty procedures.

Navigation system 30 is well known in the art, and generally includes a monitor for displaying an image of one or more body elements, such as portions of the knee 38 of patient 34. The image is generated from an image data set stored within a computer, and the image data set is typically obtained from preoperative computed tomography (CT) or magnetic resonance image (MRI) scan. The image data set includes reference points for at least one body element which have a fixed spatial relation relative to the body element. These reference points may be sensors attached to the body, or sensors on an auxiliary frame which is fixed with respect to the body. The position sensing unit 28 may be a sensor array or digitizer for identifying, during the surgical procedure, the position of the reference points, and the computer modifies the image data set during the procedure according to the positions of each of the reference points. These and other types of surgical navigation systems are well known in the art and will not be further described herein.

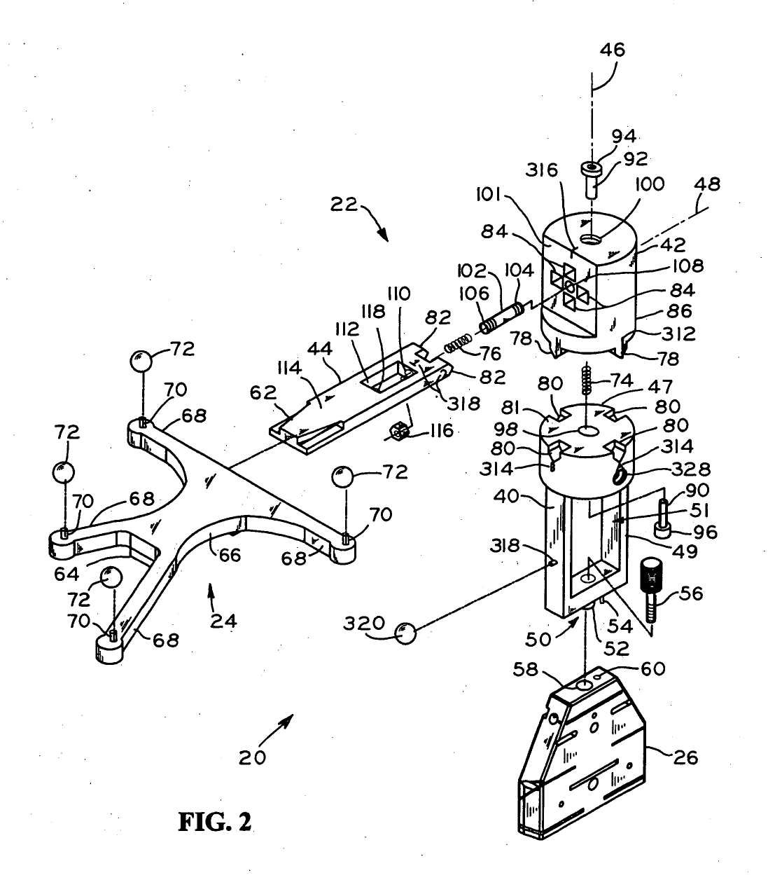

Referring to FIGS. 1 and 2, support arm 22 includes first member or portion 40, second member or portion 42, and third member or portion 44. First member 40 and third member 44 are pivotably coupled to second member 42. In one alternative embodiment, first member 40 and third member 44 are coupled to second member 42 in a manner allowing translation of first member 40 relative to second member 42 as well as translation of third member 44 relative to second member 42. Support arm 22 may include fewer or more than three members or portions that form a variable geometry in one or more degrees of freedom. In support arm 22, first member 40 may be pivotably coupled to second member 42 such that second member 42 is pivotably adjustable relative to first member 40. Specifically, second member 42 rotates about first axis 46, which, in one exemplary embodiment, is defined by the longitudinal axis of second member 42. Similarly, third member 44 may be pivotably coupled with second member 42 so that third member 44 is rotatable about second axis 48, which is defined by the longitudinal axis of third member 44. In the exemplary embodiment, second axis 48 is substantially perpendicular to first axis 46.

Referring to FIG. 2, first member 40 includes cylindrical portion 47 and frame 49 having opening 51. Distal to cylindrical portion 47, frame 49 includes a surgical instrument mounting interface 50 for coupling surgical instrument 26, for example a cut guide, to support arm 22. First mounting interface 50 provides a universal mount for multiple types of instruments. For example, exemplary first mounting interface 50 includes two noncoaxial bosses 52 and 54 which are engageable in mating bores or recesses 58 and 60 of instrument 26, and a threaded screw 56 is inserted through boss 52 of interface 50 and is threaded into recess 58 of instrument 26 to secure instrument 26 to first member 40. Advantageously, first mounting interface 50 and mating recesses 58 and 60 are formed so that support arm 22 may be repeatably precisely coupled to instrument 26 and/or other instruments in a predefined geometry with respect to support arm 22 and tracking array 24.

A second mounting interface 62 is located on third member 44 for coupling tracking array 24 to support arm 22. Second mounting interface 62 may be a universal mount for repeatably precisely coupling tracking array 24 to support arm 22 in a predefined geometry. In the exemplary embodiment, second mounting interface 62 is a dovetail projection which is receivable by dovetail receptacle 64 of body 66 of tracking array 24 so that tracking array 24 may be repeatably precisely coupled to support arm 22 in a predefined geometry with respect to support arm 22 and instrument 26.

Tracking array 24 includes body 66 having arms or projections 68 projecting therefrom. The end of each projection 68 distal from body 66 includes a mounting interface, for example, posts 70 (FIG. 2) for coupling reference elements 72 to tracking array 24. Reference elements 72 are detectable by position sensing unit 28 (FIG. 3) of computer-assisted navigation system 30. Many types of reference elements usable in computer-assisted surgical navigation systems are known. For example, the reference elements may be “active” reference elements which emit a signal to position sensing unit 28 for detection, or may be “passive” reference elements which reflect signals emitted from position sensing unit 28 for detection. In particular, active reference elements include light emitting diodes (“LED's”), or may generate acoustic, magnetic, electromagnetic, or radiologic signals. In the exemplary embodiment, reference elements 72 are passive, in the form of spherical reflectors arranged in a nonlinear planar pattern. The exemplary embodiment includes four reference elements 72; however, greater or fewer reference elements may be utilized.

In order to minimize or eliminate the need for repeated calibration after adjustment of tracking apparatus 20, first member 40 and third member 44 are movably coupled such that they may be repositioned relative to second member 42 in predetermined positions defining the relative location and relative orientation between the components. Specifically, in the exemplary embodiment, the pivotable joints about axes 46 and 48 provided between first member 40 and second member 42, and between third member 44 and second member 42, are biased together by springs 74 and 76, respectively, so that projections 78 of second member 42 engage with recesses 80 of first member 40, and projections 82 of third member 44 engage with recesses 84 of second member 42 to place tracking apparatus 20 in one of a number of predetermined positions.

In one exemplary embodiment, second member 42 is substantially cylindrical, and includes four equilaterally spaced V-shaped projections 78 projecting longitudinally from the circumference of lower portion 86 of second member 42. First member 40 includes substantially cylindrical portion 47 having recesses 80 defined adjacent the circumference of top surface 88 of first member 40. Recesses 80 correspond to the spacing and shape of projections 78. Because projections 78 and recesses 80 include four equilaterally spaced pairs, the exemplary embodiment includes four possible predetermined rotational positions in which first member 40 may be engaged with, and located with respect to, second member 42.

The rotational coupling of first member 40 and second member 42 is supported by cylindrical sleeves 90 and 92 which engage one inside the other from oppositely faced ends of bore 98 of first member 40 and bore 100 of second member 42. Bores 98 and 100 are located coaxially with first axis 46, which extends longitudinally and centrally through first member 40 and second member 42.

Heads 96 and 94 of sleeves 90 and 92 restrict relative translation of members 40 and 42. Bores 98 and 100 may be countersunk to accommodate heads 96 and 94. Spring 74 is located within the hollow central lengths of cylinders 90 and 94 and is welded or otherwise secured at its opposite ends to heads 94 and 96, thus providing a biasing force to compress first member 40 against second member 42, maintaining the engagement of projections 78 within recesses 80.

In order to rotationally reposition second member 42 relative to first member 40, second member 42 may be manually translated away from first member 40 along first axis 46 sufficient to disengage projections 78 from recesses 80. Upon disengagement of projections 78 from recesses 80, second member 42 may be rotated about axis 46 relative to first member 40 until the desired predetermined position between members 42 and 40 is achieved. Upon release of second member 42, the bias of spring 74 re-engages projections 78 within recesses 80.

In exemplary support arm 22, third member 44, which may be substantially rectangular, is coupled to second member 42 in a fashion similar to that of members 40 and 42. However, along with the coupling between first and second members 40 and 42, other forms of coupling and mechanisms for adjustment may also be utilized. Third member 44 includes V-shaped projections 82 on an end thereof opposite second mounting interface 62 to which tracking array 24 is attached. Projections 82 are engagable within complementary-shaped recesses 84 defined within substantially flat wall 101 of second member 42. Specifically, four recesses 84 provide four predefined positions of engagement for second and third members 42 and 44 in 90° increments about second axis 48.

Projections 82 of third member 44 may be biased into engagement with recesses 84 of second member 42 by spring 76. Spring 76 is located within cylinder 102. Cylinder 102 includes threads 104 and 106, located at opposite ends thereof. Thread 104 is fastened in threaded receptacle 108 located in second member 42 and centered on axis 48. Thread 106 of cylinder 102 extends through bore 110 in third member 44, and into opening 112 which extends from face 114 through third member 44. Retaining nut 116 is fastened on threads 106 of cylinder 102 to retain third member 44 to second member 42. However, cylinder 102 is of sufficient length to allow third member 44 to slide along axis 48 against the bias of spring 76 and away from second member 42, disengaging projections 82 from recesses 84 so that third member 44 may be rotated about axis 48 relative to second member 42. Spring 76 may be anchored to cylinder 102 adjacent threads 104 and, at an opposite end of spring 76, to anchor 118 located in opening 112 of third member 44. Spring 76 pulls anchor 118 toward second member 42, thereby biasing projections 82 into engagement within recesses 84, while allowing for disengagement and rotation of members 44 and 42 when desired.

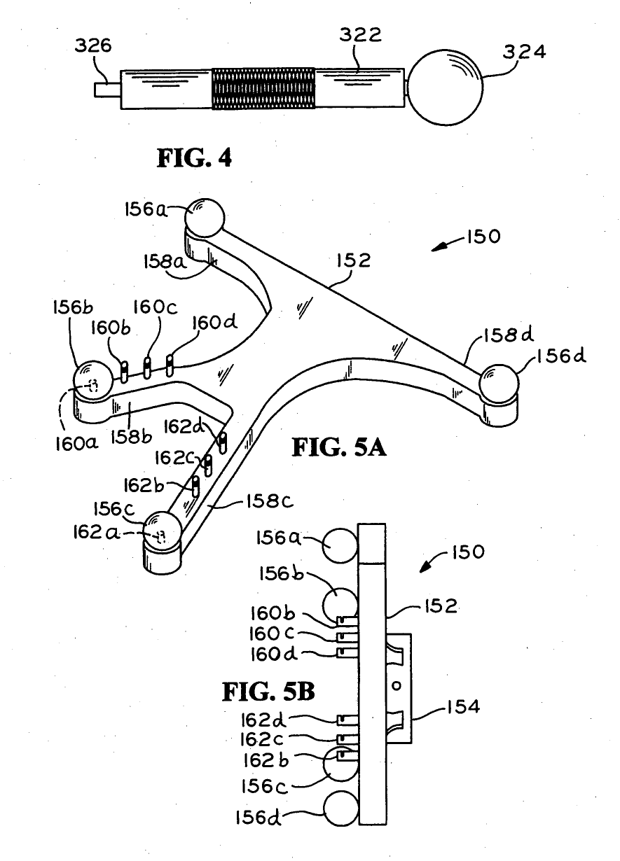

Indicators 312 and 316 located on member 42, and labels 314 and 318 located on first member 40 and third member 44, respectively, may be used to indicate, as described below, the predetermined positions of members 40, 42 and 44 relative to one another. Similarly, reference element 320 mounted on post 319 of first member 40, and recess or indentation 328 on first member 40, which is sized to receive engagement feature 326 of probe 322 (illustrated in FIG. 4 and discussed in detail below) may, in conjunction with navigation system 30, also serve the same function. Advantageously, after an initial registering or calibration of tracking apparatus 20 with navigation system 30, the foregoing features allow the position of second member 42 with respect to first member 40, as well as the position of third member 44 and tracking array 24 with respect to second member 42, to be adjusted without the need to re-register or re-calibrate tracking apparatus 20 with navigation system 30.

Although support arm 22 includes rotatable couplings, alternative mechanisms for moving each member of support arm 22 relative to the other members of support arm 22 may be utilized. For example, a pivoting or translating member, a worm gear, or other known couplings and mechanisms may be utilized. Additionally, although projection and recess pairs 78, 80 and 82, 84 provide a limited number of predetermined positions between members 40 and 42 and between members 44 and 42, other mechanisms may be utilized that provide fewer or additional predefined positions between the members.

Referring to FIGS. 5A and 5B, a second, alternative tracking array 150 includes body 152, mounting interface 154, and reference elements 156a-156d. Tracking array 150 may be coupled to support arm 22 in the manner described above with reference to tracking array 24 to track an instrument, such as instrument 26 shown in FIGS. 1-3, in computer-assisted navigation system 30 (FIG. 3) relative to anatomical structures of patient 34.

Advantageously, reference elements 156a-156d may be arranged in various nonlinear patterns to enable navigation system 30 to distinguish between multiple tracking arrays 150 and in turn, to distinguish between various different instruments to which arrays 150 may be coupled. Reference elements 156a-156d may be active or passive reference elements, as described above, the positions of which are detectable by position sensing unit 28 (FIG. 3). At least one of reference elements 156a-156d is adjustably movable relative to the remaining reference elements so that various distinguishable patterns of elements 156a-156d may be utilized.

Second exemplary tracking array 150 includes reference elements 156b and 156c which are movable along projections 158b and 158c of body 152, respectively. Specifically, posts 160a-160d and 162a-162d may be used to selectively position reference elements 156b and/or 156c relative to reference elements 156a and 156d, which are mounted on projections 158a and 158d, respectively. For example, reference element 156b is shown mounted on post 160a; however, reference element 156b may also be mounted on any one of posts 160b, 160c and 160d. Likewise, reference element 156c is shown mounted on post 162a; however, reference element 156c may also be mounted on any one of posts 162b, 162c and 162d. Reference elements 156b and 156c may be retained on posts 160a-160d and 162a-162d by conventional hardware, for example, threads, or by a mechanical detent or a press fit, for example.

The number of movable reference elements 156a-156d and mounting posts 160a-160d and 162a-162d may be selected as desired and located on one or more of projections 158a-158d. Tracking array 150 includes four possible mounting positions for each of reference elements 156b and 156c, thereby providing sixteen unique patterns of reference elements 156a-156d that may be distinguished by computer-assisted navigation system 30. The pattern of reference elements 156a-156d identify and aid tracking of each configuration of array 150 and instrument to which the array is coupled, independently of any other configurations of array 150 and other associated instruments. Computer-assisted navigation system 30 may be programmed to automatically recognize the instrument associated with a particular reference element pattern, or may be manually instructed to do so by the surgeon or other operator prior to or during a surgical procedure.

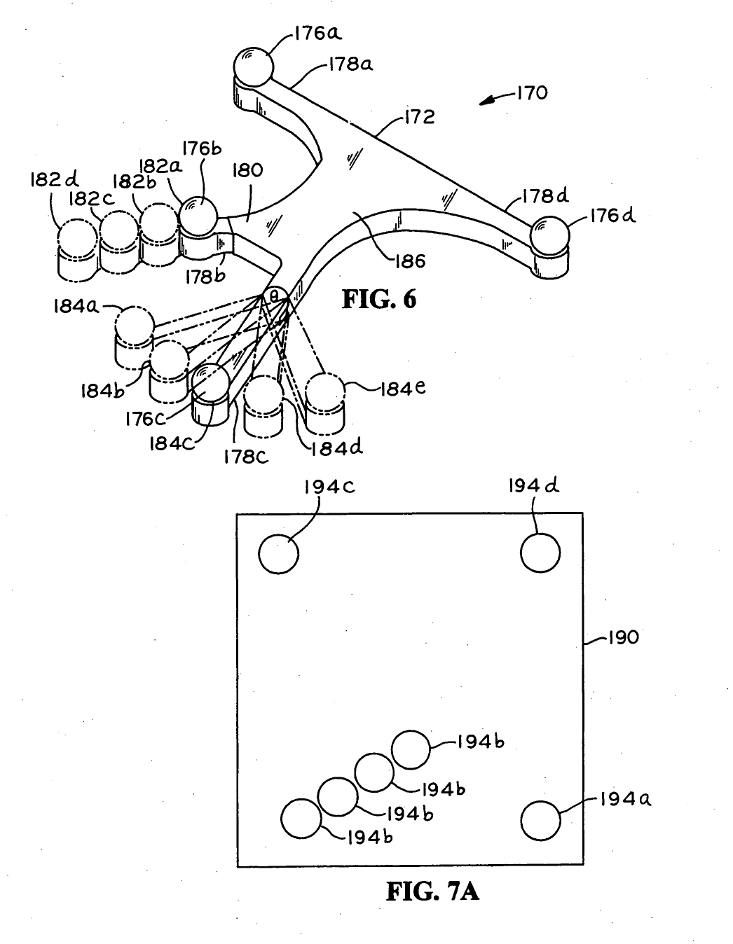

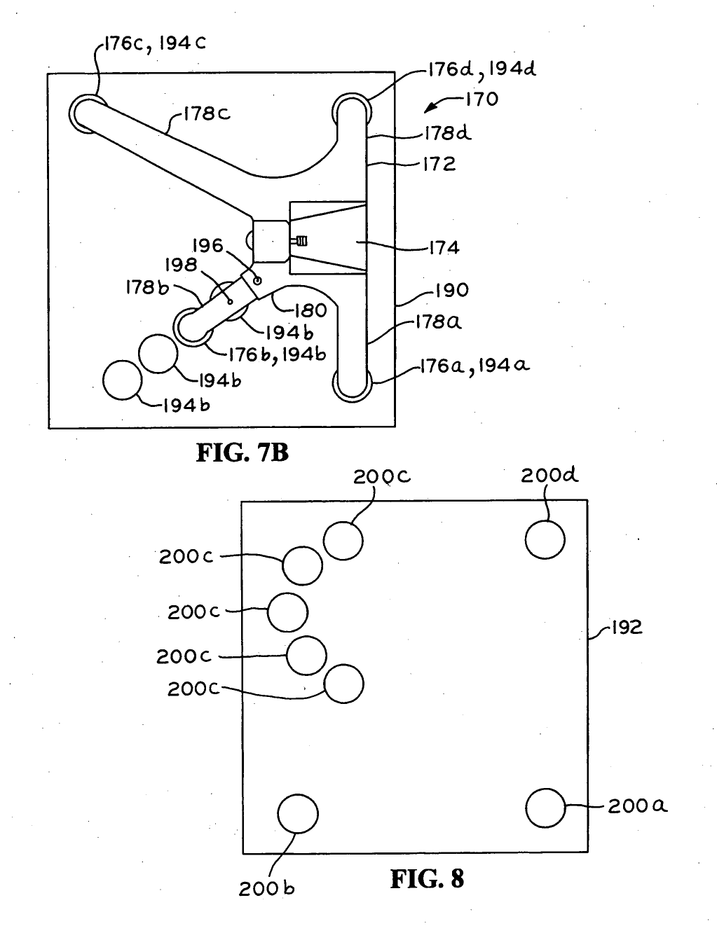

Referring to FIG. 6, another alternative tracking array 170 includes body 172, mounting interface 174 (FIG. 7B) and reference elements 176a-176d. At least one of reference elements 176a-176d of tracking array 170 is movable relative to the remaining reference elements. For example, third exemplary tracking array 170 may include one or both of slidably coupled adjustment projection 178b and pivotably coupled adjustment projection 178c, which include movable reference elements 176b and 176c, respectively.

Adjustment projection 178b may slidably engage sleeve 180 defined in body 172. Projection 178b is thereby capable of extending outwardly relative to body 172, for example to predefined positions 182a-182d shown in FIG. 6. By repositioning reference element 176b relative to the remaining reference elements, computer-assisted navigation system 30 (FIG. 3) is enabled to distinguish between various configurations of tracking array 170 and the instrument to which each tracking array is coupled. Similarly, adjustment projection 178c may be pivotably coupled to body 172 such that reference element 176c may be moved, for example between the predefined positions 184a-184e shown in FIG. 6.

Referring to FIGS. 7A and 8, templates 190 and 192 may be utilized to accurately reposition and calibrate the position of movable reference elements 176b or 176c. For example, template 190 (FIG. 7A) is designed for a tracking array having a slidably coupled adjustment projection 178b, and template 192 (FIG. 8) is designed for a tracking array having a pivotably coupled adjustment projection 178c. Other templates accommodating both a slidably coupled projection and a pivotally coupled projection are also contemplated.

For example, referring to FIG. 7B, tracking array 172, including fixed reference element 176c and movable reference element 176b, may be placed in template 190. Specifically, upper face 186 (FIG. 6) of array 170 is faced toward template 190 and reference elements 176a-176d are positioned in their respective calibration bores 194a-194d. Template 190 includes four bores 194b for positioning reference element 176b. Bores 194b correspond to the four exemplary predefined positions 182a-182d of reference element 176b.

Referring to FIG. 7B, sleeve 180 of array 170 may include device 196 for securing adjustment projection 178b relative to array body 172. For example, device 196 may be a set screw, a ball and spring, or other positioning device or fastener for securing adjustment projection 178b relative to array 172. Also, device 196 may engage one of a plurality of recesses 198 defined along the length of support projection 178b. Advantageously, recesses 198 enable device 196 to accurately position support projection 178b and therefore reference element 176b in a predefined position relative to array body 172 so that calibration of array 170 after adjustment may not be required.

Referring to FIG. 8, template 192 may be similarly utilized to locate reference element 176c and adjustable projection 178c of tracking array 170 having pivotably adjustable reference element 176c. Specifically, reference elements 176a-176d may be located in the respective matching ones of bores 200a-200d. Five bores 200c are provided to correspond to predefined positions 184a-184e of reference element 176c. Adjustable projection 178c may be fixed in a selected position with respect to tracking array 170 in a similar manner as those described above with respect to adjustment projection 178b.

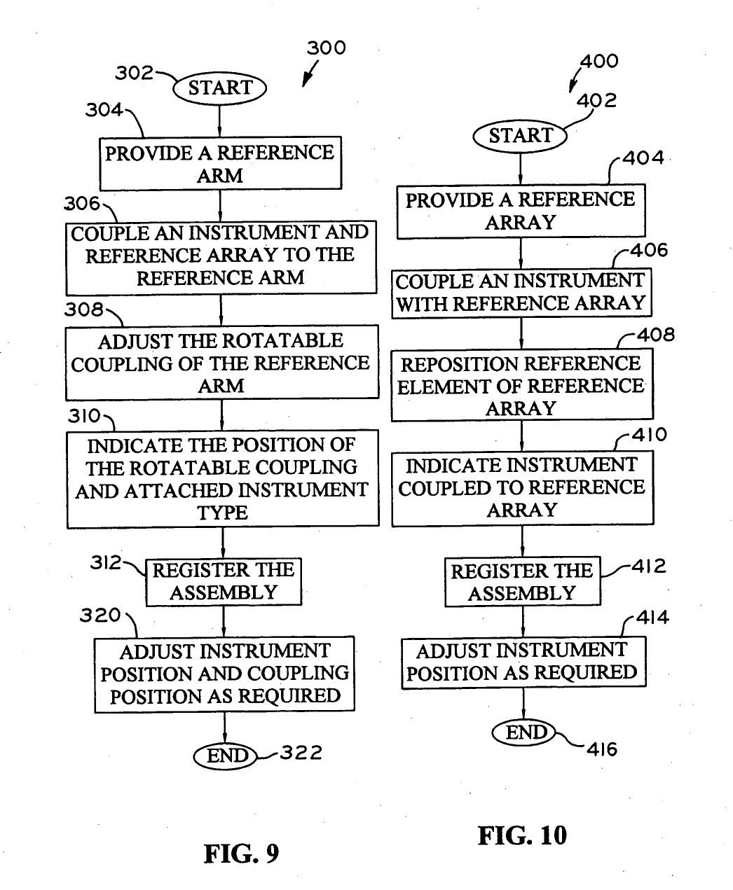

Referring to FIG. 9, an exemplary method 300, which begins at step 302, includes steps for using computer-assisted navigation system 30 to position an instrument, for example instrument 26 (FIGS. 1 and 3), with respect to anatomical structures of patient 34. In step 304, a support arm is provided, for example, support arm 22 of FIG. 1, having at least one rotatable adjustment coupling, a mounting interface for instrument 26, and a tracking array 24, 150 or 170 (FIGS. 1, 5A and 6).

In step 306, tracking apparatus 20 is assembled by coupling instrument 26 to support arm 22 using mounting interface 50 shown in FIG. 2, for example, and by coupling tracking array 24, 150 or 170 to support arm 22 using mounting interface 62 shown in FIG. 2, for example. The tracking array may be coupled to support arm 22 either before or after instrument is coupled to tracking arm 22.

In step 308, the surgeon adjusts the rotatable coupling(s) of support arm 22 as desired. For example, first element 40 and third element 44 may be rotatably repositioned relative to second element 42 to provide a desired geometry between support arm 24, 150 or 170 and instrument 26 to allow easier access to particular anatomical structures during the surgical procedure, or to more accurately position the tracking array within the field of detection of position sensing unit 28.

In step 310, indication of the relative position of the rotatable couplings and the type of instrument 26 utilized are indicated to computer-assisted navigation system 30 (FIG. 3). Indicating step 310 may occur by preprogramming navigation system 30 to recognize that a particular reference element pattern of tracking array 24, 150 or 170 indicates a particular type of instrument is in use, for example instrument 26. Alternatively, the surgeon or other operator may manually enter the type of instrument associated with tracking array 24, 150 or 170 into the computer of navigation system 30 using a keyboard, graphic pointer, touch screen, or similar input device. Similarly, the positions of third member 44 and first member 40 relative to second member 42 of support arm 22 may be indicated manually by the operator. For example, first indicator 312 (FIG. 1) of second member 42 may be located adjacent one of labels 314 of first member 40, and second indicator 316 may be located adjacent one of labels 316 of third member 44. Thus, the appropriate labels 314 and 316 which relate to the predefined geometry of instrument 26 relative to tracking array 24, 150 or 170 may be manually entered in navigation system 30 by the operator.

Alternatively, navigation system 30 may be preprogrammed to automatically recognize the predefined positions of support arm 22. First member 40 may include a mounting post 319, or another mounting interface, for coupling reference element 320 to first member 40. Reference element 320 is detectable by position sensing unit 28 so that navigation system 30 may determine the geometric relationship between tracking array 24, 150 or 170 and first member 40 and thereby automatically recognize the predefined position of support arm 22.

Another alternative method of indicating the position of the rotatable couplings of support arm 20 is to utilize reference probe 322, shown in FIG. 4. Reference probe 322 includes reference element 324 and engagement feature 326. Engagement feature 326 may be positioned in recess 329 (FIG. 1) of first member 40 to allow navigation system 30 to determine the position of first member 40 relevant to tracking array 24, 150 or 170, and thus the relevant geometry of instrument 26 to tracking array 24, 150 or 170.

In step 312, tracking apparatus 20, including support arm 22, tracking array 24, 150 or 170, and instrument 26, is registered in navigation system 30. Computer implemented image guidance systems which provide for the registration of an actual anatomical structure with a three dimensional model representing that structure, together with the registration or localization of another object such as a surgical instrument within the image coordinate system to facilitate the display of the relative positions of the object and the actual anatomical structure are well known in the art, and thus will not be described in detail herein. Registration enables navigation system 30 to track and to assist in the positioning of instrument 26 relevant to anatomical structures of patient 34.

In step 320, the position of instrument 26 with respect to the anatomical structures of patient 34 may be adjusted as required. Additionally, support arm 22 may be adjusted as required to reposition tracking array 24, 150 or 170 relative to instrument 26. For example, the surgeon may require tracking array 24, 150 or 170 to be moved to allow for unobstructed access to an anatomical structure of patient 34. Alternatively, tracking array, 24, 150 or 170 may require repositioning relative to instrument 26 in order to provide an improved orientation for detection by position sensing unit 28.

Further, after instrument 26 is positioned with respect to the anatomical structures of patient 34, instrument 26 may be secured with other instrumentation (not shown) to a patient anatomical structure, and tracking array 24, 150 and 170 and support arm 22 may then be uncoupled from instrument 26. Method 300 is complete in step 322.

Method 400, illustrated in FIG. 10, includes steps for identifying an instrument, for example instrument 26 shown in FIGS. 1 and 3, in computer-assisted navigation system 30. Method 400 begins in step 402.

In step 404, a tracking device, for example tracking array 150 or 170 (FIG. 5A and 6) having reference elements 156a-156d and/or 176a-176d, is provided. In step 406, the tracking array is coupled with instrument 26. Advantageously, tracking array 150 or 170 includes reference element 156b or 176b which is movable relative to the remaining reference elements, thereby providing a distinct reference element pattern or configuration which is distinguishable from other configurations of tracking arrays 150 or 170 by navigation system 30.

In step 406, the operator couples instrument 26 with tracking array 150 or 170. Tracking array 150 or 170 may be coupled directly to instrument 26, or may include a support arm, such as support arm 22 described above. In step 408, reference element 156b or 176b is repositioned with respect to tracking array body 152 or 172, as discussed above.

In step 410, navigation system 30 receives an indication of the type of instrument 26 coupled to tracking array 150 or 170. This indication may be in the form of an operator manually identifying the instrument type to navigation system 30, or by navigation system 30 being preprogrammed to associate a particular instrument type with a particular reference element pattern, as determined by the position of movable reference element 156b and 176b relevant to the remaining reference elements.

In step 412, the assembly consisting of at least tracking array 150 or 170 and instrument 26 may be registered with a navigation system 30. Registration enables navigation system 30 to track and to determine and guide the position of instrument 26 via the tracking of tracking array of 150 or 170 by reference device 28.

In step 414 the surgeon adjusts the position of instrument 26 as desired relative to the anatomical structures of patient 34. Method 400 is complete in step 416.

While this invention has been described as having exemplary embodiments, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A tracking apparatus for use with a surgical instrument in a computer-assisted surgical navigation system, comprising:
a support arm, including a first member having a surgical instrument mounting interface, and a second member adjustably coupled to said first member; and
a tracking array adjustably coupled to said second member, said tracking array including at least one reference element which is registerable in the navigation system.

2. The tracking apparatus of claim 1, wherein at least one of said first member and said second member, and said second member and said tracking array, are adjustable with respect to one another between a plurality of selectable, predefined positions.

3. The tracking apparatus of claim 1, wherein at least one of said first member and said second member, and said second member and said tracking array, are rotationally adjustable with respect to one another.

4. The tracking apparatus of claim 3, wherein said first member and said second member are rotationally adjustable about a first axis, and said second member and said tracking array are rotationally adjustable about a second axis different from said first axis.

5. The tracking apparatus of claim 1, wherein said first and second members are rotatable and translatable relative to one another, and said adjustable coupling therebetween further comprises:
a plurality of recesses formed in one of said first member and said second member;
a plurality of projections formed on the other of said first member and said second member, said projections respectively engageable within said recesses; and
a biasing member biasing said first and second members toward one another.

6. The tracking apparatus of claim 1, wherein said second member and said tracking array are rotatable and translatable relative to one another, and said adjustable coupling therebetween further comprises:
a plurality of recesses formed in one of said second member and said tracking array;
a plurality of projections formed on the other of said second member and said tracking array, said projections respectively engageable within said recesses; and
a biasing member biasing said second member and said tracking array toward one another.

7. The tracking apparatus of claim 1, wherein at least one of said first member, said second member, and said tracking array includes a reference indicator which is registerable in the navigation system.

8. The tracking apparatus of claim 1, wherein said tracking array includes a plurality of said reference elements, each said reference element registerable in the navigation system.

9. The tracking apparatus of claim 8, wherein at least one of said reference elements is adjustably coupled to said tracking array, whereby the relative position of said at least one reference element with respect to others of said reference elements may be varied.

10. The tracking apparatus of claim 9, wherein said at least one of said reference elements is adjustably coupled to said tracking array via one of:
a pivotally adjustable connection; and
a linearly adjustable connection.

11. A tracking apparatus for use with a surgical instrument in a computer-assisted surgical navigation system, comprising:
a support arm including a surgical instrument mounting interface; and
a tracking array adjustably coupled to said support arm, said tracking array including a plurality of reference elements which are registerable in the navigation system, at least one of said reference elements adjustably coupled to said tracking array whereby the relative position of said at least one reference element with respect to others of said reference elements may be varied.

12. The tracking apparatus of claim 11, wherein said at least one reference element is adjustably coupled to said tracking array via one of:
a pivotally adjustable coupling; and
a linearly adjustable coupling.

13. The tracking apparatus of claim 11, wherein said tracking array includes a plurality of mounting locations associated with said at least one reference element, said at least one reference element selectively attachable to said tracking array at selected ones of said mounting locations, whereby the relative position of said at least one reference element with respect to others of said reference elements may be varied.

14. The tracking apparatus of claim 11, wherein said support arm further comprises:
a first member including said surgical instrument mounting interface; and
a second member to which said tracking array is adjustably coupled, said second member adjustably coupled to said first member.

15. The tracking apparatus of claim 14, wherein said first member and said second member are rotationally adjustable about a first axis, and said second member and said tracking array are rotationally adjustable about a second axis different from said first axis.

16. The tracking apparatus of claim 14, wherein at least one of said first member, said second member, and said tracking array includes a reference indicator which is registerable in the navigation system.

17. A tracking array for use with a surgical instrument in a computer-assisted surgical navigation system, comprising:
a body member; and
a plurality of reference elements coupled to said body member, said reference elements registerable in the navigation system, at least one reference element adjustably coupled to said body member, whereby the relative position of said at least one reference element with respect to others of said reference elements may be varied.

18. The tracking array of claim 17, wherein said body member includes a plurality of arms projecting therefrom, said plurality of reference elements respectively coupled to said arms.

19. The tracking array of claim 18, wherein at least one of said arms is pivotally coupled to said body member, whereby the relative position of at least one reference element associated with said arm may be varied with respect to others of said reference elements.

20. The tracking array of claim 18, wherein at least one of said arms is linearly adjustable with respect to said body member, whereby the relative position of at least one reference element associated with said arm may be varied with respect to others of said reference elements.

21. The tracking array of claim 17, wherein said body member includes a plurality of mounting locations associated with said at least one reference element, said at least one reference element attachable to selected ones of said mounting locations whereby the relative position of said at least one reference element with respect to others of said reference elements may be varied.

22. A tracking apparatus for use with a surgical instrument in a computer-assisted surgical navigation system, comprising:
a support arm including a surgical instrument mounting interface;
a tracking array adjustably coupled to said support arm, said tracking array including at least one reference element which is registerable in the navigation system; and
said support arm and said tracking array further including cooperating reference indicators registerable in the navigation system for determining a relative orientation between said support arm and said tracking array.

23. The tracking apparatus of claim 22, wherein said support arm further comprises:
a first member including said surgical instrument mounting interface; and
a second member to which said tracking array is adjustably coupled, said second member adjustably coupled to said first member.

24. The tracking apparatus of claim 23, wherein said first member and said second member are rotationally adjustable about a first axis, and said second member and said tracking array are rotationally adjustable about a second axis different from said first axis.
